(19) United States
(12) Patent Application Publication
(10) Pub. No.: US 2015/0320589 A1
(43) Pub. Date: Nov. 12, 2015

(54) HEATING TOOL

(71) Applicant: Kobayashi Pharmaceutical so., Ltd., Osaka-shi (JP)

(72) Inventors: Daisuke Nishioka, Osaka-shi (JP); Hiromichi Tanaka, Osaka-shi (JP)

(21) Appl. No.: 14/410,320

(22) PCT Filed: Jun. 27, 2013
(86) PCT No.: PCT/JP2013/067608

§ 371 Date: Dec. 22, 2014

(30) Foreign Application Priority Data
Jun. 29, 2012 (JP) 2012-147849

(51) Int. Cl. A61F 7/03

(52) U.S. Cl. CPC A61F 7/03; A61F 2007/0226

(57) ABSTRACT

Provided is a heating tool which is capable of providing less cool feeling at the time of attaching to the skin, warming the skin effectively and supplying oil effectively to the skin, as well as providing less cool feeling after peeling from the skin and providing a high warming effect even after use. This heating tool is a heating tool including an adhesive layer, wherein the adhesive layer has a specific heat of less than 3 J/g·K at 20° C., the adhesive layer includes a base and oil with a specific heat of less than 3 J/g·K at 20° C. and a boiling point of 100° C. or higher at 1 atmospheric pressure, and the oil is contained in an amount of 30 to 90 wt % in the adhesive layer.

### TECHNICAL FIELD

The present invention relates to a heating tool comprising an adhesive layer. More particularly, the present invention relates to a heating tool which provides less cool feeling at the time of attaching the adhesive layer to the skin, provides a high warming effect and effectively supplies oil to the skin as well as provides less cool feeling after peeling the adhesive layer from the skin.

### BACKGROUND ART

Conventionally, heating tools such as disposable hand warmers have been used as a warming tool for the body and a thermotherapy tool. In particular, disposable hand warmers are frequently used because they are excellent in portability, safety, convenience and the like, and they are inexpensive. In common disposable hand warmers, an exothermic composition which generates heat in the presence of air is used, and this heat generating mechanism provides a heat retaining effect. Conventionally, heating tools have been reported which can be directly attached to the skin.

For example, Patent document 1 discloses a heat generating sheet agent which comprises an aqueous adhesive layer containing polyacrylic acid and/or polyacrylate, a cellulose derivative, and a polyvalent metallic compound and having a ball tack of 15 or more is provided. Patent document 1 discloses that the aqueous adhesive layer has excellent usability such that the aqueous adhesive layer is hardly peeled and turned-up at the time of attaching to the skin, is not slacked even in the state of high temperature by close sealing, can be clearly peeled after use, and has less skin irritation.

Patent document 2 discloses a steam-generating composition which is applied to the skin or the mucous membrane, wherein the composition releases steam to the skin or the mucous membrane and the released steam and heat allows an agent or a cosmetic component to be effectively absorbed through the skin.

As described above, heating tools have been reported which can be directly attached to the skin and have improved sense of use, however, Patent document 1 has disadvantages that cool feeling is easily provided at the time of attaching a heat generating sheet agent to the skin or after peeling it from the skin, and it relatively takes time to warm the agent. Furthermore, Patent document 2 has disadvantages that a steam-generating composition is difficult to be closely attached to the skin, and therefore, thermal sensing is difficult to be provided unless a temperature at which the steam-generating composition generates heat is set relatively high, an agent or a cosmetic component is difficult to be supplied effectively to the skin, and further, since heat is lost from the skin by vaporization heat of water at the time of removing the steam-generating composition from the skin, cool feeling is easily provided, and accordingly, a warming effect is not sustained.

As described above, even in these days when heating tools which can be directly attached to the skin have already been described, more excellent heating tools are needed.

### PRIOR ART DOCUMENT

### Patent Documents

### Patent Document 1: Japanese Patent Laid-open Publication No. 2001-224621

### Patent Document 2: Japanese Patent No. 3049707

### SUMMARY OF THE INVENTION

### Technical Problems

From the above description, an object of the present invention is to provide a heating tool which can provide less cool feeling at the time of attaching to the skin, effectively warm the skin and effectively supply oil to the skin, as well as provide less cool feeling after peeling from the skin and provide a high warming effect to the skin even after use.

### Solutions to the Problems

The present inventors have intensively studied in order to solve the problems, and have found that when a heating tool comprises an adhesive layer having a specific heat of less than 3 J/g·K at 20° C. and containing 30 to 90 wt % of oil having a specific heat of less than 3 J/g·K at 20° C. and a boiling point of 100° C. or higher at 1 atmospheric pressure, the tool can provide less cool feeling at the time of attaching to the skin, effectively warm the skin and effectively supply the oil to the skin, as well as provide less cool feeling after peeling from the skin and provide a good sustained warming effect to the skin even after use. The present invention has been accomplished by conducting further investigations based on such findings.

That is, the present invention provides the following aspects.

Item 1: A heating tool comprising an adhesive layer, wherein

the adhesive layer has a specific heat of less than 3 J/g·K at 20° C.,

the adhesive layer comprises a base and oil with a specific heat of less than 3 J/g·K at 20° C. and a boiling point of 100° C. or higher at 1 atmospheric pressure, and the oil is contained in an amount of 30 to 90 wt % in the adhesive layer.

Item 2: The heating tool according to item 1, wherein the heating tool is directly attached to the skin through the adhesive layer.

Item 3: The heating tool according to item 1 or 2, wherein the oil is contained in an amount of 40 to 90 wt % in the adhesive layer.

Item 4: The heating tool according to any one of items 1 to 3, wherein the oil is at least one selected from the group consisting of hydrocarbon, silicone oil, vegetable oil, animal oil, glycerin fatty acid ester, fatty acid, aliphatic alcohol, aromatic alcohol, polyhydric alcohol, ether, and ester.

Item 5: The heating tool according to any one of items 1 to 4, wherein the base is at least one selected from the group consisting of a rubber-based adhesive, an acrylic-based adhesive, a silicone-based adhesive, and a urethane-based adhesive.

Item 6: The heating tool according to any one of items 1 to 5, wherein the adhesive layer further comprises an active component.

Item 7: The heating tool according to item 6, wherein the active component is a moisturizing component and/or a warming sensation component.

Item 8: The heating tool according to any one of items 1 to 7, wherein the heating tool further comprises a fragrance.

Item 9: A method for warming the skin, comprising the step of attaching the heating tool according to any one of items 1 to 8 to the skin.

Item 10: A method for providing less cool feeling at the time of attaching to the skin, peeling from the skin and/or after peeling from the skin, comprising the step of attaching the heating tool according to any one of items 1 to 8 to the skin and/or the step of peeling the heating tool from the skin.

Item 11: A method for supplying oil to the skin, comprising the step of attaching the heating tool according to any one of items 1 to 8 to the skin.

Item 12: A method for relieving discomfort feeling at the time of using and/or peeling the heating tool according to any one of items 1 to 8, comprising the step of attaching the heating tool to the skin and/or the step of peeling the heating tool from the skin.

Item 13: The heating tool according to any one of items 1 to 8, wherein the heating tool is used in a method for warming the skin.

Item 14: The heating tool according to any one of items 1 to 8, wherein the heating tool is used in a method for providing less cool feeling at the time of attaching to the skin, peeling from the skin and/or after peeling from the skin.

Item 15: The heating tool according to any one of items 1 to 8, wherein the heating tool is used in a method for supplying oil to the skin.

Item 16: The heating tool according to items 1 to 8, wherein the heating tool is used in a method for reliving discomfort feeling at the time of using and/or peeling the heating tool.

### Advantageous Effects of the Invention

According to the present invention, less cool feeling can be provided at the time of attaching a heating tool comprising an adhesive layer to the skin and less cool feeling can be also provided after peeling the heating tool from the skin. The present invention can warm the skin effectively, sustain an excellent warming effect not only in applying the heating tool to the skin, but also after peeling the heating tool from the skin, and in addition, supply oil to the skin effectively. The present invention can relieve discomfort feeling such as tightness and stiffness of the skin at the time of using and peeling the heating tool.

### BRIEF DESCRIPTION OF THE DRAWINGS

FIG. 1 is an exemplification of a heating tool comprising an adhesive layer, and shows a model view of the heating tool in which an exothermic composition is housed in a container bag having air permeability and the adhesive layers are laminated in one portion of the container bag. In FIG. 1, the heating tool in which the adhesive layers are laminated is housed in an outer bag having non-air permeability.

FIG. 2 is an exemplification of a heating tool comprising an adhesive layer, and shows a model view of the heating tool in which an exothermic composition is housed in a container bag having air permeability and the adhesive layers are laminated in one portion of the container bag. In FIG. 2, the heating tool in which the adhesive layers are laminated is housed in an outer bag having non-air permeability. The shape shown in FIG. 2 is one example of a heating tool suitably applied for an eye portion.

FIG. 3 is a graph showing results as compared with the warming effect of each heating tool comprising an adhesive layer.

### MODE FOR CARRYING OUT THE INVENTION

The heating tool comprising an adhesive layer of the present invention has a feature in that the adhesive layer has a specific heat of less than 3 J/g·K at 20° C., the adhesive layer comprises a base and oil with a specific heat of less than 3 J/g·K at 20° C. and a boiling point of 100° C. or higher at 1 atmospheric pressure, and the oil is contained in an amount of 30 to 90 wt % in the adhesive layer. Hereinafter, the heating tool comprising an adhesive layer of the present invention will be described.

### Adhesive Layer

The heating tool of the present invention comprises an adhesive layer. The adhesive layer used in the present invention has a specific heat of less than 3 J/g·K, more preferably 0.5 to 2.7 J/g·K, and still more preferably 0.5 to 2.5 J/g·K at 20° C. The specific heat of the adhesive layer is measured by Differential Scanning Calorimetry (DSC method).

Additionally, the adhesive layer used in the present invention contains a base and oil. The oil has a specific heat of less than 3 J/g·K at 20° C. and a boiling point of 100° C. or higher at 1 atmospheric pressure. The oil has a specific heat of preferably 0.5 to 2.7 J/g·K, and more preferably 0.5 to 2.5 J/g·K at 20° C. The oil has a boiling point of preferably 120° C. or higher, and more preferably 150° C. or higher at 1 atmospheric pressure. Similarly as described above, the specific heat of the oil is also measured by Differential Scanning Calorimetry (DSC method). The boiling points of the oils are known.

Such oil is not limited as long as the specific heat and the boiling point described above are satisfied, and for example, any cosmetically or pharmaceutically acceptable oils may be used. Examples of the oils include hydrocarbons, silicone oils, vegetable oils, animal oils, glycerin fatty acid esters, fatty acids, aliphatic alcohols, aromatic alcohols, polyhydric alcohols, ethers, and esters. More particularly, examples of the oils include hydrocarbons such as liquid paraffin, isoparaffin, polybutene, vaseline, squalene, squalane, and chlorotrifluoroethylene; silicone oils including dimethyl silicone oil, methylphenyl silicone oil, cyclic dimethyl silicone oil, and modified silicone oils such as silicone oil obtained by introducing an organic chain into these silicone oils; vegetable oils such as olive oil, sesame oil, china wood oil, corn oil, soy oil, castor oil, rapeseed oil, rice oil, camellia oil, safflower oil, coconut oil, palm oil, palm kernel oil, cottonseed oil, sunflower oil, perilla oil, linseed oil, peanut oil, avocado oil, almond oil, grape seed oil, and jojoba oil; animal oils such as whale oil, shark oil, liver oil, lard oil, beef tallow oil, sheep oil, and horse oil; glycerin fatty acid esters including monoglycerides such as glyceryl monostearate, glyceryl monopalmitate, glyceryl monooleate, glyceryl monolaurate, and glyceryl monocaprylate, diglycerides such as cytidine diphosphate diglyceride, diglyceride palmitate, and diglyceride oleate, and triglycerides such as tricaprin, trilaurin, trimyristin, tripalmitin, and tristearin; fatty acids such as caprylic acid, undecanoic acid, lauric acid, tridecanoic acid, myristic acid, pentadecanoic acid, palmitic acid, margaric acid, stearic acid, and oleic acid; aliphatic alcohols such as capryl alcohol, lauryl alcohol, myristyl alcohol, cetyl alcohol, stearyl alcohol, and oleyl alcohol; aromatic alcohols such as benzyl alcohol, salicyl alcohol, anise alcohol, vanillyl alcohol, veratryl alcohol, cuminyl alcohol, phenethyl alcohol, hydroxybenzyl alcohol, α-cumyl alcohol, and cinnamyl alcohol; polyhydric alcohols including polyglycerins such as glycerin, diglycerin, triglycerin and tetraglycerin, ethylene glycol, diethylene glycol, triethylene glycol, polyethylene glycol (PEG200, PEG600, PEG1000, PEG2000, PEG6000, PEG20000, etc.), propylene glycol (PPG250, PPG700, PPG1000, PPG2000, PPG4000, etc.), and polybutylene glycol (PBG500, PBG700, PBG1000, PBG2000, etc.); ethers including polyethylene glycol glyceryl ethers such as polyoxyethylene glycol monomethyl ethers (400, 1000, 2000), polyoxyethylene dimethyl ether (550), and polyoxyethylene glycol glyceryl ethers (450, 750), polypropylene glycol glyceryl ethers such as polyoxypropylene diglyceryl ether (700), glycol ethers such as ethylene glycol monomethyl ether, diethylene glycol monomethyl ether, triethylene glycol monomethyl ether, ethylene glycol monoisopropyl ether, diethylene glycol monoisopropyl ether, ethylene glycol monobutyl ether, diethylene glycol monobutyl ether, ethylene glycol monoisobutyl ether, diethylene glycol monoisobutyl ether, ethylene glycol monohexyl ether, diethylene glycol monohexyl ether, ethylene glycol monophenyl ether, diethylene glycol monophenyl ether, ethylene glycol monobenzyl ether, diethylene glycol monobenzyl ether, propylene glycol monomethyl ether, dipropylene glycol monomethyl ether, tripropylene glycol monomethyl ether, propylene glycol monopropyl ether, dipropylene glycol monopropyl ether, propylene glycol monobutyl ether, dipropylene glycol monobutyl ether, propylene glycol monophenyl ether, diethylene glycol dimethyl ether, diethylene glycol methylethyl ether, and ethylene glycol dibutyl ether; esters such as myristic acid methyl ester, palmitic acid methyl ester, steatic acid methyl ester, n-nonadecanoic acid methyl ester, stearic acid ethyl ester (α), n-nonadecanoic acid ethyl ester, and arachic acid ethyl ester.

Examples of the oils include preferably oils which satisfy the specific heat and the boiling point described above and are liquid oils at 35° C., more preferably liquid oils at 20° C., and still more preferably liquid oils at 0° C.

Examples of the oils include preferably hydrocarbons, silicone oils, and vegetable oils, and more preferably liquid paraffin, isoparaffin, polybutene, vaseline, squalene, squalane, chlorotrifluoroethylene; dimethyl silicone oil, methylphenyl silicone oil, cyclic dimethyl silicone oil, and modified silicone oils such as silicone oil obtained by introducing an organic chain into these silicone oils; olive oil, sesame oil, china wood oil, corn oil, soy oil, castor oil, rapeseed oil, rice oil, camellia oil, safflower oil, coconut oil, palm oil, palm kernel oil, cottonseed oil, sunflower oil, perilla oil, linseed oil, peanut oil, avocado oil, almond oil, grape seed oil, and jojoba oil.

These oils may be used alone or in combination of two or more kinds.

The proportion of the oil in the adhesive layer is, for example, 30 to 90 wt %, preferably 40 to 90 wt %, more preferably 50 to 90 wt %, and still more preferably 60 to 85 wt %.

The adhesive layer used in the present invention further contains a base. The base is not limited as long as it is innocuous even in contact with the skin and the adhesive layer can exhibit adhesive properties to such an extent that the adhesive layer can be attached to the skin when the base is combined with the above oils. Examples of the base include those conventionally used as adhesive components in a patch to the skin, etc., and are not limited as long as the desired effects are provided. Thus, the bases include conventionally known adhesive components.

Example of the base include rubber-based adhesives such as polystyrene-polybutadiene-polystyrene copolymer (SBS), polystyrene-polyisoprene-polystyrene copolymer (SIS), and polystyrene-polyethylene-polybutylene-polystyrene copolymer (SEBS), and polystyrene-polyethylene-polypropylene-polystyrene copolymer (SEPS), serving as a hydrogenated copolymer of these, acrylic-based adhesives such as methyl acrylate (MA), ethyl acrylate (EA), 2-ethylhexyl acrylate (HA), and butyl acrylate (BA), silicone-based adhesives such as addition curing type and peroxide curing type adhesives, and urethane-based adhesives such as ether type and ester type adhesives. Examples of the base include preferably rubber-based adhesives, and more preferably polystyrene-polybutadiene-polystyrene copolymer (SBS), polystyrene-polyisoprene-polystyrene copolymer (SIS), and polystyrene-polyethylene-polybutylene-polystyrene copolymer (SEBS), and polystyrene-polyethylene-polypropylene-polystyrene copolymer (SEPS), serving as a hydrogenated copolymer of these.

These may be used alone or in combination.

The amount of the base is not limited as long as the desired effects are provided, but the proportion of the base in the adhesive layer is, for example, 5 to 70 wt %, more preferably 10 to 60 wt %, and still more preferably 10 to 40 wt %.

The adhesive layer used in the present invention may further contain active components, as necessary. Examples of the active components include, but are not particularly limited to, moisturizing, thermal sensing (promoting blood circulation), whitening, anti-wrinkle, anti-aging, anti-inflammatory, antibacterial, antioxidant, slimming, cleansing, and relaxing components, and the adhesive layer may contain components having the desired effects depending on purpose.

Examples of the active components include ceramide, hyaluronic acid, squalane, collagen, and Lipidure (registered trademark), an action of which is moisturizing. Furthermore, examples of the active components include capsaicin, caffeine, tocopherol nicotinate, and nonylic acid vanillylamide, an action of which is thermal sensing. Examples of the other active components include vitamin A, retinol, palmitic acid, retinol, ascorbyl tetrahexyldecanoate, tocopherol, dl-α-tocopherol, dl-α-tocopherol acetate, L-menthol, camphor, sulfur, pyridoxine hydrochloride, sorbitol, maltitol, hyaluronic acid, phellodendron bark extract, glycyrrhiza extract, aloe extract, seaweed extract, carrot extract, mulberry extract, and licorice extract, an action of which is conventionally known. Additionally, for example, a component to be blended in a conventional poultice, an antipyretic component, a component having an effect for alleviating shoulder stiffness, etc. are mentioned as active components, and any components may be used without particular limitation depending on purpose. These components may be used alone or in combination of two or more kinds depending on purpose.

The amount of the active component is not limited as long as the desired effects are provided, but when the active component is contained, the proportion of the active component in the adhesive layer is, for example, 0.0001 to 20 wt %, more preferably 0.001 to 10 wt %, and still more preferably 0.005 to 5 wt %.

The adhesive layer may further contain any components such as a preservative, a dye, a pigment, a fragrance and others, as necessary, as long as the desired effects are provided. Examples of the fragrance include those described below.

As used herein, the term “comprise” also includes both the meaning of “consist essentially of” and the meaning of “consist of”.

In the present invention, the adhesive layer may be included in at least one portion of the heating tool, as described below. The thickness, size, shape or the like of the adhesive layer included in the heating tool is not limited as long as the effects of the present invention are provided, and may be appropriately set depending on the purpose of use such as portions to which the heating tool comprising an adhesive layer is applied. From the viewpoint of providing the effects of the present invention effectively, the thickness of the adhesive layer is, for example, 1 to 3000 μm, and more preferably 10 to 300 μm. The size of the adhesive layer may be appropriately set depending on the application portions such as eyes, shoulder, waist, back, arm, leg and sole. The shape thereof is not limited as long as the effects of the present invention are achieved, and may be appropriately set depending on the application portion or the like for the usability sake, as necessary.

The heating tool comprising an adhesive layer of the present invention is not limited as long as the effects of the present invention are provided, and may be, for example, directly attached to the skin through the adhesive layer, or may be attached onto clothes or the like through the adhesive layer. From the viewpoint of achieving the desired effects effectively, the heating tool comprising an adhesive layer of the present invention is directly attached to the skin through the adhesive layer.

As described above, the adhesive layer used in the present invention has the specific heat described above and contains the certain oil at a certain proportion. When the adhesive layer is used, it can provide less cool feeling at the time of attaching the adhesive layer to the skin or peeling the adhesive layer from the skin as well as after peeling from the skin, and in addition, the adhesive layer can be quickly warmed through the heating effect by the heating tool, and the skin can be effectively warmed. From the above, the warming effect allows pores of the skin to easily open and allows oil and active components blended in the adhesive layer to be easily supplied to the skin. The oil blended in the adhesive layer can cover the skin to thereby prevent sweat from evaporating from the surface of the skin, and therefore, the warming effect can be favorably sustained in attaching the adhesive layer to the skin as well as even after peeling from the skin. As described above, when the adhesive layer contains oil, discomfort feeling such as tightness and crunchy in using and peeling the adhesive layer can be relieved. When the oil itself contained in the adhesive layer has an effect of promoting blood circulation, etc., effects derived from the oil can be also achieved together. When additional active components are blended in the adhesive layer, effects derived from the additional active components can be also achieved together. The adhesive layer is hardly peeled and turned-up in attaching to the skin, is not slacked, and can be peeled from the skin without remaining the base on the skin.

### Heating Tool

In the present invention, the heating tool comprising the adhesive layer is not limited as long as the effects of the present invention can be achieved, and conventionally known heating tools which can generate heat may be used. From the viewpoint of being excellent in portability, safety, convenience and the like, for example, conventionally known heating tools which can generate heat in the presence of oxygen may be used, and for example, exothermic compositions containing an oxidizable metal powder, a water soluble salt, and water, or exothermic compositions further containing a component such as an oxidation accelerator or a water retaining agent can be used. Although not limiting the present invention, one example of the heating tools will be described below which comprises an exothermic composition comprising an oxidation accelerator, an oxidizable metal powder, a water soluble salt, a water retaining agent, and water and in which at least the exothermic composition is housed in a container bag having air permeability. Other heating tools can be prepared and produced with reference to the following description.

Examples of the oxidation accelerator include, but are not limited to as long as the desired effects are provided, carbon black, graphite, activated carbon, coal, charcoal, bamboo charcoal, black lead, acetylene black, and coffee grounds charcoal. These may be used alone or in combination of two or more kinds.

The amount of the oxidation accelerator is not limited as long as the desired effects are provided, but the proportion of the oxidation accelerator in the exothermic composition is, for example, 1 to 30 wt %, preferably 3 to 25 wt %, and more preferably 5 to 23 wt %.

The amount of the oxidation accelerator relative to the oxidizable metal powder described below is not also limited as long as the desired effects are provided, but the amount of the oxidation accelerator per 100 parts by weight of the oxidizable metal powder is, for example, 1 to 30 parts by weight, preferably 2 to 25 parts by weight, and more preferably 5 to 20 parts by weight.

Examples of the oxidizable metal powder contained in the exothermic composition include, but are not limited to as long as it is a metallic powder which generates heat when being oxidized, iron powder, aluminum powder, magnesium powder, zinc powder, and copper powder, and preferably include iron powder. Examples of the iron powder include reduced iron powder, cast iron powder, atomized iron powder, and electrolytic iron powder. These may be used alone or in combination of two or more kinds. Although not limited as long as the desired effects are provided, the oxidizable metal powder has an average particle size of, for example, 0.01 to 1000 μm, preferably 0.1 to 500 μm, and more preferably 0.5 to 300 μm from the viewpoint of comfort when the heating tool is attached to the body, or heat generation efficiency, etc. The average particle size of the oxidizable metal powder can be measured by a JIS method using a standard sieve, and the like.

The amount of the oxidizable metal powder is not limited as long as the desired effects are provided, but the proportion of the oxidizable metal powder in the exothermic composition is, for example, 20 to 80 wt %, preferably 30 to 70 wt %, and more preferably 40 to 65 wt %.

The water soluble salt contained in the exothermic composition are blended in order to promote the oxidation of the oxidizable metal powder, and example thereof are not limited as long as the desired effects are provided, and include hydrochlorides and sulfates of alkali metals such as sodium and potassium, hydrochlorides and sulfates of alkaline earth metals such as calcium and magnesium, and hydrochlorides and sulfates of metals such as iron, copper, aluminum, zinc, nickel, silver, and barium. More preferable examples thereof include potassium chloride, sodium chloride, and the like. These may be used alone or in combination of two or more kinds.

The amount of the water soluble salt is not also limited as long as the desired effects are provided, but the proportion of the water soluble salt in the exothermic composition is, for example, 0.1 to 10 wt %, preferably 0.5 to 7 wt %, and more preferably 1 to 5 wt %.

The water retaining agent has a function of retaining water, and examples thereof are not limited as long as it has the function and the desired effects are provided, and include porous substances, water absorbing resins, and the like. Examples of the water retaining agent include natural and synthetic inorganic substances such as vermiculite, perlite, calcium silicate, kaolin, talc, smectite, mica, bentonite, calcium carbonate, silica gel, alumina, zeolite, silicon dioxide, and diatomaceous earth; and natural and synthetic organic substances such as pulp, wood flour (sawdust), cotton, polyacrylate-based resin, polysulfonate-based resin, maleic anhydride salt-based resin, polyacrylamide-based resin, polyvinyl alcohol-based resin, polyethylene oxide-based resin, polyaspartate-based resin, polyglutamate-based resin, polyalginate-based resin, starches, and celluloses. Preferable examples thereof include vermiculite, perlite, wood flour (sawdust), diatomaceous earth, perlite, silica gel, aluminum oxide, and the water absorbing resin. These may be used alone or in combination of two or more kinds.

The amount of the water retaining agent is not also limited as long as the desired effects are provided, but the proportion of the water retaining agent in the exothermic composition is, for example, 1 to 20 wt %, preferably 3 to 15 wt %, and more preferably 5 to 10 wt %.

In the water retaining agents, a water retaining agent having a porous structure such as vermiculite, in particular, can serve not only as a water retaining agent, but also as an air passageway.

Distilled water, tap water, ion-exchanged water, pure water, ultrapure water, industrial water, and others can be used as water.

The amount of water is not also limited as long as the desired effects are provided, but the proportion of water in the exothermic composition is, for example, 1 to 50 wt %, preferably 5 to 40 wt %, and more preferably 10 to 35 wt %.

As one aspect of the exothermic composition, examples of the proportion in the exothermic composition include, but are not limited to, 5 to 20 wt % for the oxidation accelerator, 40 to 80 wt % for the oxidizable metal powder, 3 to 15 wt % for the water retaining agent, 1 to 10 wt % for the water soluble salt, and 1 to 40 wt % for water.

In addition to the above-mentioned components, other components which can be blended in the exothermic composition may be further blended in the exothermic composition, as necessary. Examples of the components include, but are not limited to, a surfactant, a hydrogen generation inhibitor, a thickener, an excipient.

In the exothermic composition, the total amount of the oxidation accelerator, the oxidizable metal powder, the water soluble salt, the water retaining agent, and water is not limited as long as the desired effects are provided, and the heating temperature in the exothermic composition may be appropriately set so as to be a temperature suitable for applying the composition to the skin. The temperature is, for example, 32 to 85° C., and more preferably about 40 to 70° C. (measurement values according to JIS S4100).

The exothermic composition is prepared by mixing the components described above. The exothermic composition may be prepared in the presence of oxygen, or may be prepared in vacuum or in an inert gas atmosphere. These can be prepared according to a conventionally known procedure.

The exothermic composition thus prepared is housed in a container bag having air permeability for housing the exothermic composition (hereinafter, sometimes referred to as “container bag for the exothermic composition”). In the container bag having air permeability for housing the exothermic composition, conventionally known container bags can be used without limitation as long as they can house the exothermic composition and have air permeability. For example, in consideration of prevention of leaking the exothermic composition, durability against the heat generation by the exothermic composition, and improving sense of use of heating tool, etc., air permeable bag and the like used in hitherto known disposable hand warmers can be used as the container bag for the exothermic composition.

Although not limiting the present invention, more specific examples thereof include, as the container bag for the exothermic composition, container bags which has a laminated structure in which a resin film having air permeability is laminated on a woven fabric or non-woven fabric having air permeability. In this case, the resin film having air permeability is disposed inside the container bag for the exothermic composition, and the woven fabric or non-woven fabric having air permeability is disposed outside the container bag for the exothermic composition.

Examples of the resin used in the resin film having air permeability include, but are not particularly limited to, preferably thermoplastic resins. Examples of the thermoplastic resins include polyethylene, polypropylene, polyester, polyamide, polyurethane, polystyrene, polyvinyl alcohol, polyvinyl chloride, polyvinylidene chloride, polycarbonate, ethylene-vinyl acetate copolymer, and the like. From the viewpoint of use of the heating tool as being attached to the body, polyethylene, polypropylene, ethylene-vinyl acetate copolymer, and the like are preferred as the thermoplastic resins. These may be used alone or in combination of two or more kinds.

The resin film having air permeability used in the present invention has pores for securing the air permeability in the resin film formed from the resin in at least one portion. The size of the pore is not limited as long as it can allow air to move in and out of the container bag for the exothermic composition, and it is of sufficient size to be capable of preventing leakage of the exothermic composition to the outside of the container bag. The sensible temperature in using the heating tool may be affected by the air permeability of the container bag for the exothermic composition, and therefore, the size, shape, and number of the pores may be appropriately determined in consideration of the sensible temperature of the heating tool upon usage. Means for forming pores in the resin film are conventionally known, and a conventional procedure can be followed.

Examples of the fiber materials of the woven fabric or non-woven fabric having air permeability include synthetic fibers such as nylon, vinylon, polyester, rayon, acrylic, polyethylene, polypropylene, acetate, polyvinyl chloride, and polybutylene terephthalate; natural fibers such as cotton, hemp, silk, and paper; and mixed fibers of synthetic fiber and natural fiber. From the viewpoint of sense of use, examples of the fiber materials include nylon, polyester, polypropylene and others, preferably nylon and polyester. These may be used alone or in combination of two or more kinds. The woven fabric or non-woven fabric is not limited as long as the fabric can allow air to move in and out of the container bag for the exothermic composition, and it can prevent leakage of the exothermic composition to the outside of the container bag. The weight per area of the fabric is, for example, preferably 25 to 70 g/m².

The laminate of the resin film having air permeability and the woven fabric or non-woven fabric having air permeability is not limited as long as the obtained laminate is strong enough as the container bag for the exothermic composition and secures air permeability. The laminate can be formed by, for example, a lamination method, and examples of the lamination method include methods of laminating by thermal bond, and methods of laminating using an adhesive such as a hot-melt adhesive, an acrylic-based adhesive, or a urethane-based adhesive. These laminates may be formed partially or entirely on the container bag for the exothermic composition as long as the desired effects are provided.

The container bag for housing the exothermic composition does not have to have air permeability on the entire container bag, i.e., it may have some portions that have no air permeability (non-air permeability portion). In this case, materials constituting a non-air permeability portion are not limited as long as they can house the exothermic composition and has non-air permeability, and conventionally known materials can be used. For example, as a material constituting a non-air permeability portion, a material constituting a non-air permeability portion in the container bag for the exothermic composition which is used in hitherto known disposable hand warmers can be used, in consideration of prevention of leaking the exothermic composition, durability against the heat generation by the exothermic composition, improving sense of use of heating tool, etc.

Although not limiting the present invention, specific examples of the material constituting a non-air permeability portion include resin films having no air permeability. Examples of the resin used in the resin films having no air permeability include, but are not particularly limited to, preferably thermoplastic resins. Examples of the thermoplastic resins include polyethylene, polypropylene, polyester, polyimide, polyurethane, polystyrene, polyvinyl alcohol, polyvinyl chloride, polyvinylidene chloride, polycarbonate, ethylene-vinyl acetate copolymer, and the like. From the viewpoint of use of the heating tool as being attached to the body, polyethylene, polypropylene, ethylene-vinyl acetate copolymer, and the like are preferred as the thermoplastic resins. These may be used alone or in combination of two or more kinds.

The resin films having no air permeability may have a laminated structure in which a woven fabric or non-woven fabric having air permeability is laminated on the resin film having no air permeability. Although not limited as long as the effects of the present invention are provided, as an example of this case, the resin film having no air permeability is disposed inside the container bag for the exothermic composition, and the woven fabric or non-woven fabric having air permeability is disposed outside the container bag for the exothermic composition.

Commercially available products may be used as the container bag for the exothermic composition.

The size and shape of the container bag for the exothermic composition are not also limited as long as the desired effects are provided, and may be appropriately determined depending on the purpose of use.

In the present invention, the heating tool comprising the adhesive layer may further contain a fragrance, and the fragrance may be appropriately determined depending on the use and preference and is not limited. Natural fragrances such as essence oils can be used alone or in combination, and synthetic single fragrances can be used alone or in combination. A natural fragrance and a synthetic fragrance can be arbitrarily combined to be used as a compound fragrance. Examples of the natural fragrances (essence oils) include, but are not limited to, vanilla, lavender, chamomile, rosemary, sage, citronella, ginger, ilang-ilang, eucalyptus, mint, rose, lily, lilac, jasmine, cardamom, lemon grass, yuzu, orange, lemon, lime, grapefruit, neroli, cedar wood, sandalwood, anise, caraway, amber, musk, civet, castoreum, and the like. Examples of the synthetic single fragrances include, but are not limited to, acetophenone, aldehyde C6-C16, allyl caproate, amyl cinnamic aldehyde, amyl salicylate, benzaldehyde, benzyl acetate, benzyl alcohol, borneol, camphor, cinnamic alcohol, citral, citronellal, citronellol, coumarin, damascone, dihydrolinalool, dihydromyrcenol, diphenyl oxide, ethyl-2-methyl butyrate, ethyl butyrate, eugenol, geraniol, geranyl acetate, phenylethyl alcohol, hedione, hexanol, cis-3-hexenol, α-hexyl cinnamic aldehyde, isoamyl acetate, lilial, limonene, linalool, linalyl acetate, 1-menthol, methylbenzoate, methyl ionone, methyl salicylate, nerol, α-pinene, β-pinene, rose oxide, terpineol, γ-nonalactone, γ-undecalactone, vanillin, and the like. Since aromatic properties can be enhanced by the heat generated in the heating tool, as the fragrance, more preferred is a fragrance exhibiting its effect in the presence of air at a temperature at which the exothermic composition generates heat, and still more preferred is a fragrance exhibiting its effect at around 32 to 85° C., and more preferably at around 40 to 70° C. The fragrance may be in the form of liquid, solid, or the like. When the fragrance is a fragrance having a relaxing effect, such as lavender or chamomile, the heating tool is said to have a relaxing effect. The effects (functions) of the fragrances are conventionally known.

The amount of the fragrance in the heating tool is not limited as long as the desired effects are provided, and the amount of the fragrance is, for example, 0.0001 to 5 parts by weight, preferably 0.01 to 1 part by weight, and more preferably 0.05 to 0.6 parts by weight per 100 parts by weight of the exothermic composition.

The manner the fragrance is included in the heating tool is not limited as long as its aroma can be imparted to the heating tool, and the fragrance may be further housed in the container bag having air permeability or may exist outside the container bag. More specifically, regarding the manner the fragrance is included in the heating tool, for example, the fragrance may be mixed with the components of the exothermic composition and housed in the container bag; the fragrance may be included in at least one portion of the container bag for the exothermic composition; or the fragrance may be included in another sheet or housed in another container bag having air permeability, etc., in advance, and the sheet, the container bag or the like may be disposed inside and/or outside the container bag for the exothermic composition.

When the fragrance is mixed with the components in the exothermic composition, for example, the fragrance itself may be mixed with the components; a mixture obtained by mixing water etc., with the fragrance by using a surfactant or the like may be mixed with the components; the fragrance or the mixture may be encapsulated in advance in conventionally known microcapsules, and the obtained encapsulated microcapsules may be mixed with the components; or the fragrance or the mixture may be supported on a carrier and then mixed with the components. From the viewpoint of preventing adhesion of the fragrance to the components in the exothermic composition as much as possible, in particular, from the viewpoint of preventing adhesion of the fragrance to the pro-oxidant and the oxidizable metal powder as much as possible, for example, the fragrance is preferably supported on a carrier in advance, and then mixed with the components. Examples of the carrier include, but are not limited to as long as the effects of the present invention are not hindered, silica, vermiculite, perlite, fluorite, zeolite, fine silicon dioxide, pulp, plastic, rubber, elastomer, and the like. The particle size of the carrier is not also limited as long as the effects of the present invention are not hindered, and examples of the average particle size is around 0.1 to 3000 μm, preferably around 0.5 to 1000 μm, and more preferably around 1 to 500 μm. The amount of the carrier is not also limited as long as the effects of the present invention are not hindered.

When the fragrance is supported on the carrier, for example, the fragrance may be supported on the components contained in the exothermic composition. From the viewpoint of the influence on heating, the fragrance is preferably supported on components other than the oxidation accelerator and the oxidizable metal powder in the exothermic composition. For example, the fragrance may be supported on a component such as the water retaining agent contained in the exothermic composition.

As described above, when the fragrance is contained in at least one portion of the container bag for the exothermic composition, for example, the fragrance may be included in such a manner that the container bag is impregnated with the fragrance in advance, or may be included in such a manner that the fragrance is kneaded into at least one of the film, the woven fabric, and the non-woven fabric constituting the container bag in advance. Also, the fragrance may be included in such a manner that the fragrance is encapsulated in microcapsules, and the microcapsules are attached to at least one of the film, the woven fabric, and the non-woven fabric constituting the container bag.

Even when the fragrance is included and even when any components are further appropriately included as necessary, the exothermic composition and the heating tool are prepared according to a conventionally known procedure, in the same manner as described above.

### Heating Tool Comprising Adhesive Layer

In the present invention, the adhesive layer may be disposed in at least a part of the heating tool, and a position and a method for including the adhesive layer in the heating tool are not limited as long as the effects of the present invention are provided. For example, the heating tool comprising the adhesive layer of the present invention is provided by including the adhesive layer in at least one part of the container bag in which the exothermic composition is housed. In this case, the container bag for housing the heating composition has air permeability as described above, however, the container bag does not have to have air permeability on the entire container bag, that is, some portions thereof may have no air permeability. From the above, the adhesive layer may be included in a portion having air permeability, or may be included in a portion having no air permeability, or may be included in both of the container bag for housing the exothermic composition. For example, the adhesive layer may be included in the heating tool in such a manner that a woven fabric, a non-woven fabric, paper etc., is laminated on the container bag for housing the exothermic composition, and the laminated woven fabric, non-woven fabric, paper etc., is disposed between the container bag and the adhesive layer.

The adhesive layer may be provided in only one portion or in a plurality of portions, i.e., two portions or more of the heating tool. As described above, the thickness, size, shape and the like of the adhesive layer included in the heating tool are not also limited as long as the effects of the present invention are provided, and may be appropriately set depending on purpose of use such as portions to which the heating tool comprising the adhesive layer is applied.

In the present invention, methods for including the adhesive layer in the heating tool are not limited as long as the adhesive layer can be provided in the heating tool, and the adhesive layer may be laminated on the heating tool, for example, by hot melt method or calendar method.

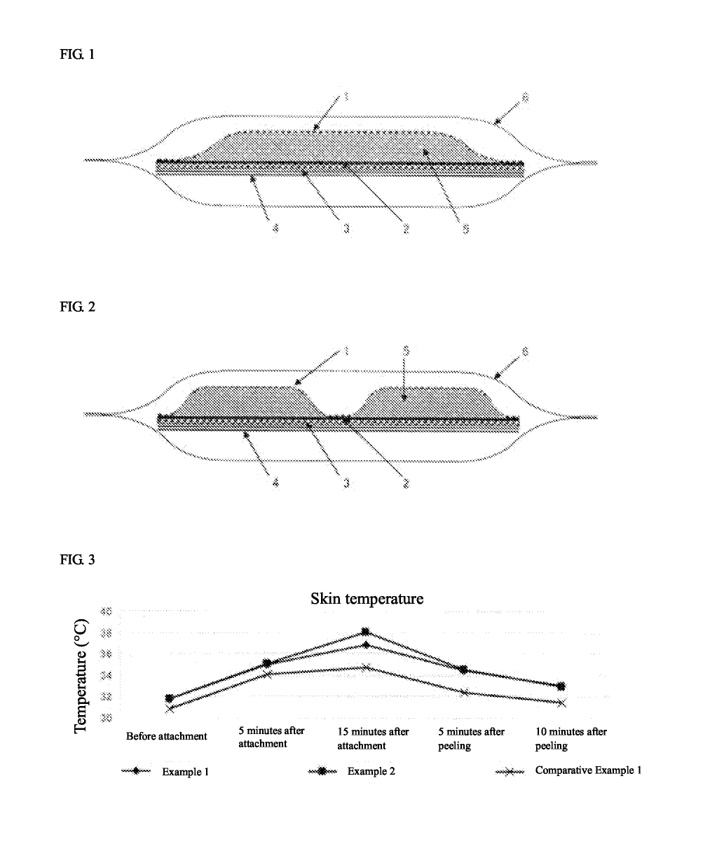

FIGS. 1 and 2 show a heating tool comprising an adhesive layer. For example, the shape shown in FIG. 2 is an example of the heating tool of the present invention which is suitably applied for an eye portion.

The heating tool comprising the adhesive layer of the present invention thus produced is generally provided and stocked in a state that it is further packaged in an outer bag having non-air permeability, in which oxygen does not permeate, to keep air-tightness. Importantly, the heating tool is stocked so as not to generate heat until use, i.e., so as not to be brought into contact with oxygen, since the exothermic composition generates heat by being brought into contact with oxygen. Then, the heating tool comprising the adhesive layer of the present invention may be used in such a manner that the outer bag is opened at the time of use, and the heating tool is taken out of the outer bag and is brought into contact with oxygen to generate heat. The outer bag used herein is not particularly limited as long as it is an outer bag having non-air permeability in which oxygen does not permeate.

The heating tool comprising the adhesive layer of the present invention thus provided is not limited as long as the effects of the present invention are provided, and for example, may be directly attached to the skin through the adhesive layer or may be attached onto clothes or the like through the adhesive layer. From the viewpoint of achieving the desired effects effectively, the heating tool comprising the adhesive layer of the present invention is directly attached to the skin through the adhesive layer. Furthermore, the portions to which the heating tool comprising the adhesive layer of the present invention is applied are not also limited, and for example, the heating tool is applied to any portions such as eyes, shoulder, waist, back, arm, leg and sole.

The heating tool comprising the adhesive layer of the present invention can provide less cool feeling at the time of attaching the adhesive layer to the skin or peeling the adhesive layer from the skin, and after peeling from the skin. The adhesive layer is quickly warmed by the heating of the heating tool, and therefore, the skin can be effectively warmed. The warming effect allows pores of the skin to easily open and allows oil and active components blended in the adhesive layer to be easily supplied to the skin, and therefore, the permeation effect of the desired components to the skin can be improved. The oil blended in the adhesive layer can cover the skin to thereby prevent sweat from evaporating from the surface of the skin, and therefore, the warming effect produced from the heating tool can be favorably maintained in applying the adhesive layer as well as even after peeling. In particular, the oil attached onto the surface of the skin after peeling can prevent evaporation heat produced by the evaporation of sweat, resulting in sustaining warm feeling. As described above, when the adhesive layer contains oil, discomfort feeling such as tightness and crunchy in using and peeling the adhesive layer can be relieved. When the oil itself contained in the adhesive layer has an effect of promoting blood circulation, etc., effects derived from the oil can be also achieved together. When additional active components are blended in the adhesive layer, effects derived from the additional active components can be also achieved together. The adhesive layer is hardly peeled and turned-up in attaching to the skin, is not slacked, and can be peeled from the skin without remaining the base on the skin.

From the above, the present invention is also said to provide a method for warming the skin, comprising the step of attaching the heating tool comprising the adhesive layer to the skin. The present invention is also said to provide a method for providing less cool feeling at the time of attaching to the skin, peeling from the skin and/or after peeling from the skin, comprising the step of attaching the heating tool comprising the adhesive layer to the skin and/or the step of peeling the heating tool from the skin. The present invention is also said to provide a method for supplying oil to the skin, comprising the step of attaching the heating tool comprising the adhesive layer to the skin. The present invention is also said to provide a method for relieving discomfort feeling at a time of using and/or peeling the heating tool comprising the adhesive layer, comprising the step of attaching the heating tool to the skin and/or the step of peeling the heating tool from the skin. The present invention is also said to provide a method comprising providing less cool feeling as described above, while warming the skin as described above, supplying oil to the skin as described above, and/or relieving discomfort feeling as described above. The heating tool comprising the adhesive layer, the components, the amounts of the components, the production method, the application method, and the like used in these methods are described similarly as described above.

### EXAMPLES

Hereinafter, the present invention will be described below by means of Examples, but the present invention is not limited to Examples provided below.

### Example 1

Liquid paraffin (manufactured by MATSUMURA OIL CO., Ltd., boiling point at 1 atmosphere: 100° C. or higher and specific heat at 20° C.: 1.88 J/g·K) as oil and a polystyrene-polyethylene-polybutylene-polystyrene copolymer (SEBS) (produced by Kraton Performance Polymers Inc.) as a base were compatibly mixed to prepare an adhesive layer 1 containing 80 wt % of liquid paraffin and 20 wt % of SEBS. The adhesive layer 1 had a specific heat of 1.96 J/g·K at 20° C.

On the other hand, iron powder (manufactured by DOWA IP CREATION CO., LTD., product name: DKP, average particle size: 100 μm), water, activated carbon (manufactured by FUTAMURA CHEMICAL CO., LTD., product name: Taiko activated carbon, average particle size: 50 μm), vermiculite (average particle size: about 500 μm), a water absorbing resin (acrylic acid polymer partial salt crosslinked product, average particle size: 250 μm), and common salt were mixed together to prepare an exothermic composition containing 55 wt % of iron powder, 25 wt % of water, 10 wt % of activated carbon, 5 wt % of vermiculite, 3 wt % of water absorbing resin, and 2 wt % of common salt. The obtained mixture was housed in a container bag having air permeability (130×95 mm) made of a porous film to which a non-air permeable sheet was partially attached (manufactured by NITTO LIFETECH CORPORATION, product name: Breathron), and sealed the bag to obtain a heating tool 1.

The adhesive layer 1 was applied to one portion of the non-air permeable sheet of the heating tool by hot melt method to obtain a heating tool comprising the adhesive layer 1. Herein, the adhesive layer 1 had a thickness of about 200 μm.

### Example 2

An adhesive layer 2 containing 80 wt % of silicone oil (manufactured by Shin-Etsu Chemical Co., Ltd., boiling point at 1 atmosphere: 100° C. or higher, specific heat at 20° C.: 1.67 J/g·K) and 20 wt % of SEBS was prepared in the same manner as described above except that silicone oil was used as oil instead of liquid paraffin. A heating tool comprising the adhesive layer 2 was obtained in the same manner as described above using the heating tool 1. Herein, the adhesive layer 2 had a thickness of about 200 μm.

### Example 3

An adhesive layer 3 containing 60 wt % of liquid paraffin and 40 wt % of SEBS was prepared in the same manner as in Example 1 except that the content of liquid paraffin was 60 wt % and the content of SEBS was 40 wt % in the adhesive layer. The adhesive layer 3 had a specific heat of 1.86 J/g·K at 20° C. A heating tool comprising the adhesive layer 3 was obtained in the same manner as in Example 1. Herein, the adhesive layer 3 had a thickness of about 200 μm.

### Example 4

An adhesive layer 4 containing 35 wt % of liquid paraffin and 65 wt % of SEBS was prepared in the same manner as in Example 1 except that the content of liquid paraffin was 35 wt % and the content of SEBS was 65 wt % in the adhesive layer. The adhesive layer 4 had a specific heat of 1.73 J/g·K at 20° C. A heating tool comprising the adhesive layer 4 was obtained in the same manner as in Example 1. Herein, the adhesive layer 4 had a thickness of about 200 μm.

### Example 5

An adhesive layer 5 was prepared in the same manner as in Example 1 except that olive oil (boiling point at 1 atmosphere: 100° C. or higher, specific heat at 20° C.: 1.99 J/g·K) was used instead of liquid paraffin and SIS was used instead of SEBS in the adhesive layer, the content of olive oil was 75 wt % and the content of SIS was 25 wt %. A heating tool comprising the adhesive layer 5 was obtained in the same manner as in Example 1. Herein, the adhesive layer 5 had a thickness of about 180 μm.

### Example 6

An adhesive layer 6 was prepared in the same manner as in Example 1 except that soy oil (boiling point at 1 atmosphere: 100° C. or higher, specific heat at 20° C.: 1.96 J/g·K) was used instead of liquid paraffin, SBS was used instead of SEBS in the adhesive layer, the content of soy oil was 70 wt % and the content of SBS was 30 wt %. A heating tool comprising the adhesive layer 6 was obtained in the same manner as in Example 1. Herein, the adhesive layer 6 had a thickness of about 150 μm.

### Test Example 1

The heating tool comprising the adhesive layer obtained in Example 1 or that obtained in Example 2 were evaluated for warming effects. Specifically, respective heating tool comprising the adhesive layer was attached to an arm, and then skin temperatures were measured at 5 and 15 minutes after attachment. Furthermore, skin temperatures were measured at 5 and 10 minutes after peeling the heating tool comprising the adhesive layer. The skin temperatures were measured with Thermography CPA-400A manufactured by CHINO CORPORATION.

Similarly, evaluated was a commercially available heating tool which warms the skin by generating steam for warming effects as Comparative Example 1.

In Examples 1 and 2 and Comparative Example 1, each test was performed on two persons, and each average value was calculated in each Example. The results are shown in Table 3.

As is clear from FIG. 3, desirable elevation of temperature after attachment was observed in Examples 1 and 2, while in Comparative Example 1, elevation of temperature was observed for 5 minutes after being attached, but subsequent elevation of temperature was not significant, which was not satisfactory. That is, in Examples 1 and 2, it was understood that the skin can be effectively warmed and thus pores of the skin are easily opened through the warming effect, oil blended in the adhesive layer and further an active component are easily supplied to the skin, resulting in improving the permeation effect of the desirable component to the skin. In both Examples, decrease of temperature was observed after peeling from the skin, however, in Examples 1 and 2, remarkable elevation of temperature was achieved during being attached, and therefore, suitable skin temperature could be maintained for a certain period even after peeling. In particular, oil attached onto the surface of the skin prevents evaporation heat produced by evaporation of sweat, and the boiling point of the oil used is 100° C. or higher, which can prevent oil itself from evaporating, and therefore, cool feeling at the time of peeling and after peeling was suppressed, and hot feeling could be also sustained. Furthermore, the specific heat of the adhesive layer and that of the oil contained in the adhesive layer were lower, and therefore, it was understood that no cool feeling is provided at the time of attaching the adhesive layer to the skin. Additionally, in Examples 1 and 2, no discomfort feeling such as tightness and crunchy was provided at the time of attaching and peeling, and the adhesive layer was not peeled and turned-up at the time of attaching, was not slacked even when the skin temperature was elevated, and could be peeled without remaining the base on the skin.

### Test Example 2

The heating tool comprising the adhesive layer obtained in Example 1, that obtained in Example 3, or that obtained in Example 4 were evaluated for sense of use based on Table 1. Specifically, respective heating tool comprising the adhesive layer was applied to an arm for 20 minutes, and then peeled therefrom to evaluate for the contents shown in Table 1. When the average value in Table 2 is 0, it shows the most favorable sense of use. The results are shown in Table 2.

A heating tool comprising an adhesive layer produced in the same manner as in Example 1 except that the content of liquid paraffin was 20 wt % and the content of SEBS was 80 wt % in the adhesive layer was defined as Comparative Example 2. Also, a heating tool comprising an adhesive layer produced in the same manner as in Example 1 except that the content of liquid paraffin was 95 wt % and the content of SEBS was 5 wt % in the adhesive layer was defined as Comparative Example 3.

TABLE 1

| Evaluation | Contents |
|---|---|
| A | Discomfort due to sticky feeling |
| B | Slightly sticky feeling |
| C | No sticky feeling and tightness, with skin moisturized |
| D | Slightly tightness |
| E | Discomfort due to tightness |

TABLE 2

| | Sub- | Sub- | Sub- | Sub- | Sub- | Average |
|---|---|---|---|---|---|---|
| | ject A | ject B | ject C | ject D | ject E | value |
| Example 1 | C | C | C | C | C | 0 |
| Example 3 | C | C | C | C | C | 0 |
| Example 4 | D | D | D | D | D | −1 |
| Comparative | E | E | E | E | E | −2 |
| Example 2 | | | | | | |
| Comparative | A | A | A | A | A | −2 |
| Example 3 | | | | | | |

Average values were calculated with scores of −2 for A, −1 for B, 0 for C, −1 for D, and −2 for E in the above table.

As is clear from Table 2, the heating tool comprising the adhesive layer of Example 1 or that of Example 3 had desirable sense of use such that no sticky feeling and tightness was provided and the skin was moderately moisturized. The heating tool comprising the adhesive layer of Example 4 had slightly less sense of use than those of Examples 1 and 3, but it had acceptable sense of use. In contrast, in Comparative Examples 2 and 3, the tools were discomfort due to tightness or discomfort due to sticky feeling.

From the above, it was surprisingly understood that the heating tool comprising the adhesive layer of the present invention provides no discomfort due to sticky feeling by oil and no uncomfortable tightness, as well as supplies oil to the skin appropriately. From the above, according to the heating tool comprising the adhesive layer of the present invention, it was understood that oil blended in the adhesive layer and further an active component can be easily permeated to the skin, resulting in improving the permeation effect of the desired components to the skin. Furthermore, the heating tool comprising the adhesive layer of the present invention had no problems in use since no tightness and crunchy were provided as described above, and the adhesive layer was not peeled, turned-up and slacked in use.

### DESCRIPTION OF THE REFERENCE NUMERALS

1. Container bag having air permeability (portion having air permeability)
2. Container bag having air permeability (portion having non-air permeability)
3. Adhesive layer
4. Releasing paper
5. Exothermic composition
6. Outer bag having non-air permeability

1. A heating tool comprising an adhesive layer, wherein
the adhesive layer has a specific heat of less than 3 J/g·K at 20° C.,
the adhesive layer comprises a base and oil with a specific heat of less than 3 J/g·K at 20° C. and a boiling point of 100° C. or higher at 1 atmospheric pressure, and
the oil is contained in an amount of 30 to 90 wt % in the adhesive layer.

2. The heating tool according to claim 1, wherein the heating tool is directly attached to the skin through the adhesive layer.

3. The heating tool according to claim 1, wherein the oil is contained in an amount of 40 to 90 wt % in the adhesive layer.

4. The heating tool according to claim 1, wherein the oil is at least one selected from the group consisting of hydrocarbon, silicone oil, vegetable oil, animal oil, glycerin fatty acid ester, fatty acid, aliphatic alcohol, aromatic alcohol, polyhydric alcohol, ether, and ester.

5. The heating tool according to claim 1, wherein the base is at least one selected from the group consisting of a rubber-based adhesive, an acrylic-based adhesive, a silicone-based adhesive, and a urethane-based adhesive.

6. The heating tool according to claim 1, wherein the adhesive layer further comprises an active component.

7. The heating tool according to claim 6, wherein the active component is a moisturizing component and/or a warming sensation component.

8. The heating tool according to claim 1, wherein the heating tool further comprises a fragrance.

9. A method for warming the skin, comprising the step of attaching the heating tool according to claim 1 to the skin.

10. The heating tool according to claim 1, wherein the heating tool is used in the method for warming the skin.
